(19) United States
(12) Patent Application Publication
(10) Pub. No.: US 2023/0061149 A1
(43) Pub. Date: Mar. 2, 2023

(54) MULTI-SENSORS CLINICAL MEASURING DEVICE AND METHOD

(71) Applicant: BIOBEAT TECHNOLOGIES LTD., PETACH TIKVA (IL)

(72) Inventors: JOHANAN MAY, PETACH TIKVA (IL); ISRAEL SARUSSI, GANEI TAL (IL); ARIK BEN ISHAY, ZORAN (IL)

(21) Appl. No.: 17/412,570

(22) Filed: Aug. 26, 2021

(51) Int. Cl. A61B 5/0205; A61B 5/0533; A61B 5/00

(52) U.S. Cl. CPC A61B 5/02055; A61B 5/0004; A61B 5/0533; A61B 5/746; A61B 5/4815; A61B 5/6823; A61B 5/6824; A61B 5/6828; A61B 2560/0214; A61B 2560/0223; A61B 2562/0219; A61B 2562/227; A61B 2562/0271

(57) ABSTRACT

A measuring device for measuring one or more clinical parameters of a patient, including a housing having multiple sensors, the sensors including one or more cardiac or cardiovascular sensors and one or more additional sensors, the device also including electrical circuitry located in the housing and including a storage unit for storing sensors data and sensors activation rules, where the sensors activation rules dictate which of the multiple sensors is used to sample the clinical parameters, and a processor to process the sensors data, the device also including a sensors switching circuit configured to determine which sensors of the multiple sensors collect information in a given time frame in accordance with the sensors’ activation rules, and an output unit to receive signal values from the sensors and to output clinical data.

### FIELD AND BACKGROUND

The invention, in some embodiments thereof, relates to clinical parameters measurement and, more particularly, but not exclusively, to devices for noninvasive measurement of clinical parameters.

It has been a problem to maintain continual contact between photoplethysmogram (PPG) or electrogram electrodes and the skin after a day or two. Dirt, moisture, and other environmental contaminants, as well as perspiration, skin oil, and dead skin cells from the patient’s body, can get between the electrode and the skin’s surface. These factors may reduce the quality of signal recordings. Physical movements of the patient and their clothing impart various compressional, tensile, and torsional forces on the contact point of the electrodes, especially over long recording times, and an inflexibly fastened electrodes will be prone to becoming dislodged. Dislodgment may occur unbeknownst to the patient, making the blood pressure (BP) or electrogram recordings worthless. Thus, it is desired to periodically remove or replace the electrodes during a long-term BP or electrogram monitoring period.

### SUMMARY

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In one aspect of the invention a measuring device is provided for measuring one or more clinical parameters of a patient, including a housing, two or more sensors located in the housing, and including one or more cardiac or cardiovascular sensors for outputting cardiac activity values, having one or more sensing probes selected from a photoplethysmogram (PPG) sensor and an electrogram sensor, one or more additional sensors, selected from a galvanic skin response (GSR) sensor, an accelerometer, and a thermometer, and electrical circuitry located in the housing and electrically connected to the two or more sensors, the electrical circuitry including a storage unit for storing sensors data and sensors activation rules, wherein the sensors activation rules dictate which of the two or more sensors is used to sample the clinical parameters, a processor configured to process the sensors data, and a sensors switching circuit configured to determine which sensors of the two or more sensors collect information in a given time frame in accordance with the sensors activation rules, an output unit configured to receive signal values from the sensors and to output clinical data.

In some cases, the device further including a measurements storage unit, configured to store historical data received from the sensors, historical sensors activation instructions and historical clinical data. In some cases, the electrical circuitry is configured to activate the one or more cardiac or cardiovascular sensors and the one or more additional sensors when the device is set to measure the one or more clinical parameters.

In some cases, the device further includes an input unit for receiving or updating the sensors activation rules. In some cases, the sensors activation rules further comprise sensors operational range, such as measuring range, sensitivity, and signal filtering profile. In some cases, the sensors activation rules further comprise target clinical parameters, such as measuring range of values, clinical parameters to measure.

In some cases, the device further including a power source connected to the two or more sensors, and the sensors activation rules comprise activation intervals associated with a measured power condition, such that activation intervals frequency of at least one of the two or more sensors is reduced when measured power source is reduced. In some cases, the power source is a battery and the measured power source is equivalent to the level of battery charging level.

In some cases, the device is a wearable device having the housing couplable to a body organ. In some cases, the body organ is a limb or a chest.

In some cases, the device further including a communication circuit adapted to communicate with one or more remote devices, and having a receiver circuit for receiving data from the one or more remote devices. In some cases, the device further including calibration functionality adapted to receive as an input data from the one or more remote devices, to processes calibration data and to output the calibration data. In some cases, the electrical circuitry stores a biological or medical condition of the patient and rules for activating the sensors based on the biological or medical condition of the patient.

In some cases, the stored biological or medical condition comprise one or more of Acute HF, COPD level, infection (sepsis), pneumonia, sleep apnea, Hypertension, hypotension, fall, other cardio-pulmonary diseases, neurological diseases, psychological conditions, general deterioration and a combination thereof. In some cases, the electrical circuitry selects a specific biological or medical parameter to be measured based on the biological or medical condition of the patient and activates the sensors in order to measure the selected specific biological or medical parameter.

In some cases, the specific biological or medical parameter comprise one or more of systolic blood pressure, diastolic blood pressure, mean arterial pressure, pulse pressure, stroke volume, cardiac output, cardiac index, systemic vascular resistance, blood oxygen saturation, tissue oxygen saturation, respiratory rate, breathing volume, pulse rate, pulse rate variability, Heart rate, Heart rate variability (HRV), cardiac arrhythmia, level of sweat, movements, gait, calories consumption, body temperature, Hemoglobin level, glucose/sugar level, sleep quality, lactate, bilirubin level, fat level, and a combination thereof. In some cases, the electrical circuitry stores allowed range or values of the specific biological or medical parameters, where the electrical circuitry generates an alert in case one or more of the specific biological or medical parameters are outside the allowed range or values. In some cases, at least two of the two or more sensors collect information simultaneously regarding the same biological or medical parameter.

In some cases, determining which sensors of the two or more sensors will determine the value of the clinical parameter in a given time frame based on the patient’s condition and/or device operational status and/or the signals measured from the two or more sensors. In some cases, one sensor of the two or more sensors is used to calibrate measurements of a second sensor of the two or more sensors.

In some cases, the sensor activation rules dictate that a first sensor of the two or more sensors is used to measure a specific biological or medical parameter when the measured values are in a first set range and a second sensor of the two or more sensors is used to measure the specific biological or medical parameter when the measured values are in a second set range. In some cases, the device including at least five sensors, where the at least five sensors including a photoplethysmogram (PPG) sensor, an electrogram sensor, a galvanic skin response (GSR) sensor, an accelerometer, and a thermometer. In some cases, the at least five sensors collect information in a continuous manner. In some cases, the at least five sensors collect information at set intervals. In some cases, the set intervals depend on a status of a battery providing electrical power to the device.

In some cases, the clinical parameters comprise one or more of systolic blood pressure, diastolic blood pressure, mean arterial pressure, pulse pressure, stroke volume, cardiac output, cardiac index, systemic vascular resistance, blood oxygen saturation, tissue oxygen saturation, respiratory rate, breathing volume, Heart rate, Heart rate variability (HRV), cardiac arrhythmia, level of sweat, movements, gait, calories consumption, body temperature, Hemoglobin level, glucose/sugar level, sleep quality, lactate, bilirubin level, fat level, and a combination thereof. In some cases, the electrical circuitry is configured to measure signal to noise ratio of at least one of the two or more sensors and selecting which of the sensors to be used based on the signal to noise ratio.

In another aspect of the invention a method is provided for measuring one or more clinical parameters of a patient by a measuring device having two or more sensors and sensors activation rules, the method including activating one or more cardiac and/or cardiovascular sensors which are part of the two or more sensors, according to the sensors activation rules, receiving cardiac activity data from the one or more cardiac and/or cardiovascular sensors, activating one or more additional sensors which are part of the two or more sensors, according to the sensors activation rules, storing data received from the activated sensors, processing sensors signal values into clinical data by a computing unit located in the measuring device, and outputting the clinical data.

In some cases, the method further including checking the power level in the power source, and reducing the measurement frequency of one or more of the activated sensors if power level is below a predefined threshold. In some cases, the method further including receiving a biological or medical condition of the patient, generating a measurement plan based on the biological or medical condition of the patient, and obtaining sensors activation rules for the measurement plan.

In some cases, the method further including calculating a signal to noise ratio for one or more of the two or more sensors, and activating one or more sensors based on the calculated signal to noise ratio. In some cases, the activating of one or more cardiac and/or cardiovascular sensors and one or more additional sensors is by activating three or more sensors in a continuous manner. In some cases, the method further including receiving data from a second measuring device, and generating calibration data for the two or more sensors and/or the sensors activation rules. In some cases, the method further including receiving data from a first and a second sensors of the two or more sensors, comparing between the values of measurements received from the first and the second sensors, and generating calibration data for the two or more sensors and/or the sensors activation rules.

In some cases, the method further including selecting clinical condition of the patient, defining clinical parameters for monitoring by the measuring device, and obtaining activation rules based on defined one or more clinical parameters for monitoring. In some cases, the method further including obtaining sensors activation rules based on the patient’s current and/or historical condition. In some cases, the method further including obtaining sensors activation rules based on device operational status and/or the signals measured from the two or more sensors.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are schematic illustrations of a measuring device according to exemplary embodiments of the invention;

FIGS. 2A to 2D are flow diagrams of some examples of processes for measuring clinical parameters, according to some embodiments of the invention;

FIGS. 3A and 3B are flow diagrams of some examples of processes steps for measuring clinical parameters, using a second device according to some embodiments of the invention;

FIG. 4 is an illustration of a bottom view of a wearable apparatus, according to exemplary embodiments of the invention; and

FIG. 5 is an illustration an isometric view of a wearable measuring device, according to exemplary embodiments of the invention.

### DETAILED DESCRIPTION

The invention, in some embodiments thereof, relates to clinical parameters measurement and, more particularly, but not exclusively, to devices for noninvasive measurement of clinical parameters.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

According to an aspect of the invention, there a provided a device having multiple sensors for measuring patient physiological parameters simultaneously. In some examples the device has sensors and electrical circuit arrangements for continuous monitoring of the physiological parameters. In some example configurations, the devices and method are fitted to be used at home, office, clinic, or hospital. In some example configurations, the devices and method are fitted to be operated by a patient, technician, or health professional.

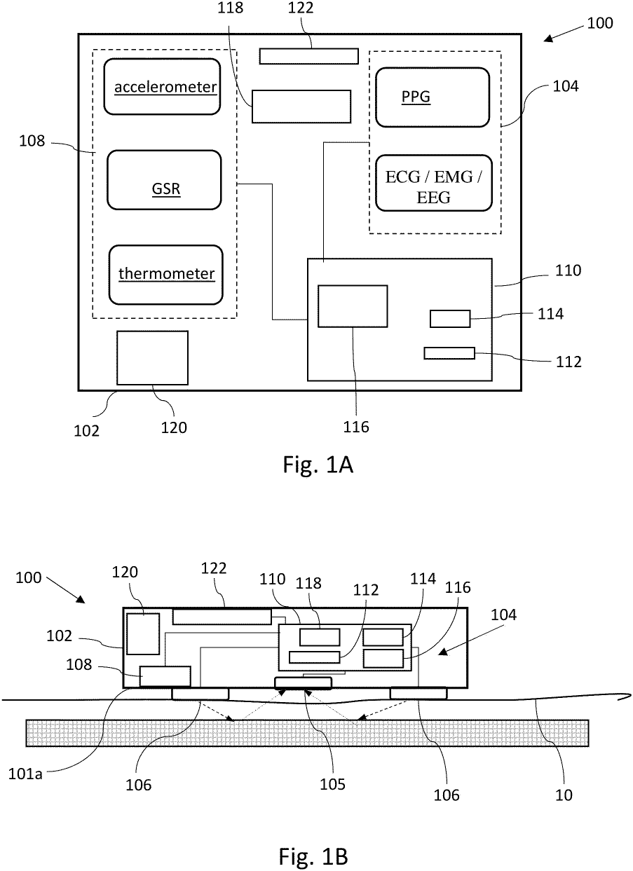

Referring now to the drawings. FIGS. 1A and 1B are schematic illustrations of a measuring device for measuring clinical parameters of a patient, according to exemplary embodiments of the invention. As shown in FIGS. 1A and 1B, measuring device 100 has a housing 102, and two or more sensors located in the housing 102. According to some embodiments, the two or more sensors, can be sub-grouped into categories of sensors having shared characteristics, such as type of measured parameters (e.g., electrical, mechanical, dynamics, optical), type of measured physical phenomenon (pressure, electrical conductivity, thermal, etc.), which clinical systems is being measured (e.g., cardiac, skin, muscular).

According to the example embodiment shown in FIGS. 1A and 1B, measuring device 100 has one or more cardiac or cardiovascular sensors 104 for outputting cardiac activity values. In some embodiments, cardiac or cardiovascular sensors 104 are selected from a photoplethysmogram (PPG) sensor and an electrogram sensor. In addition, measuring device 100 has one or more one or more additional sensors 108. In some embodiments, additional sensors 108 can be an arrangement of sensors selected to measure a variety of physical phenomenon such as a galvanic skin response (GSR) sensor, an accelerometer, and a thermometer.

According to some embodiments, measuring device 100 includes electrical circuitry 110 located in the housing 102 and electrically connected to the two or more sensors. In some embodiments, electrical circuitry 110 has a storage unit 112 for storing sensors data and sensors activation rules.

The sensors activation rules are defined to dictate which of the sensors or sub-groups of sensors is used to sample the clinical parameters. In some example embodiments, electrical circuitry 110 is configured to activate one or more cardiac or cardiovascular sensors 104 when the measuring device 100 is set to measure clinical parameters. In some example embodiments, electrical circuitry 110 is configured to activate one or more additional sensors 108 when the measuring device 100 is set to measure clinical parameters. For example, the electrical circuitry may be configured to select a specific biological or medical parameter to be measured based on the biological or medical condition of the patient and to activate at least one of the two or more sensors in order to measure the selected specific biological or medical parameter. Some examples of the selected biological or medical condition are Acute HF, COPD level, infection (sepsis), pneumonia, sleep apnea, Hypertension, hypotension, fall, general deterioration, and a combination thereof.

In some embodiments, the measuring device 100 has at least five sensors, for example a photoplethysmogram (PPG) sensor, an electrogram sensor, a galvanic skin response (GSR) sensor, an accelerometer and a thermometer. In some embodiments, the at least five sensors collect information in a continuous manner. The continuous manner may be defined as sampling measurements during a long period of time, for example at least one hour, one day, one week, one month and one year. In some embodiments, the at least five sensors collect information at set intervals. The set intervals may depend for example on a status of a battery providing electrical power to the measuring device 100.

According to some embodiments, electrical circuitry 110 has one or more processors 114 configured to process the sensors data.

In some embodiments, electrical circuitry 110 includes a sensors switching circuit 116 configured to determine which sensors of the two or more sensors collect information in a given time frame in accordance with the sensors’ activation rules. The sensors switching circuit 116 may access data stored in the storage unit 112, such as the sensors activation rules.

According to some embodiments, electrical circuitry 110 is configured to measure a signal to noise ratio of at least one of the two or more sensors. In some embodiments, the sensors activation rules are used to select the sensors in accordance with noise to ratio rate calculated based on preceding measurements received from the sensors. In some embodiments, the sensors activation rules comprise sensors operational range, such as measuring range, sensitivity, and a signal filtering profile. In some embodiments, the sensors activation rules comprise target clinical parameters, such as measuring range of values, clinical parameters to measure. In some embodiments, the sensors activation rules are associated with a biological or medical condition of the patient. In some embodiments, the sensors activation rules are used to select the sensors is in accordance with an input that includes a selection of clinical parameters to measure or a measurement plan to define required measurements by the measuring device 100.

According to some embodiments, the sensors activation rules dictate that a first sensor of two or more sensors is used to measure a specific biological or medical parameter when the measured values are within a first set range and a second sensor of the two or more sensors is used to measure the specific biological or medical parameter when the measured values are within a second set range. For example, using PPG to measure Heart rate (HR) when HR is below 100 beats per minutes (BPM) and ECG when HR is above 100 BPM. In some embodiments, at least two of the two or more sensors collect information simultaneously regarding the same medical parameter.

According to some embodiments, determining which sensors will measure the value of the clinical parameter in a given time frame is based on the patient’s condition. For example, during running, measure HR using PPG. During rest, measure HR using ECG. During coughing, for COPD patient and for HF patient suffers from having lungs congestion, use PPG to measure respiratory rate instead of ECG signal. In some embodiments, determining which sensors will measure the value of the clinical parameter in a given time frame is based on the signals measured from the one or more of the sensors.

According to some embodiments, one sensor is used to calibrate measurements of a second sensor of the sensors.

According to some embodiments, the measuring device 100 has an input unit 120. In some embodiments, input unit 120 has an interface for a person to select operational functions or operational parameters. In some embodiments, input unit 120 is adapted for receiving the sensors activation rules. In some embodiments, input unit 120 is adapted for updating the sensors activation rules. In some embodiments, input unit 120 includes a receiver for receiving data or operational parameters from a remote device.

In some embodiments, sensors data include sampled data received from one or more of the sensors. In some embodiments, data is sampled in a rate which is adjusted in accordance with a quality score associated with the sampled data. In some embodiments, quality score of the sampled data and/or adjusting the rate is by a sampling functionality associated with processor 114.

The measuring device 100 may include an output unit 122 configured to receive signal values from the sensors, the processing unit, and/or the storage unit and to output clinical data and/or clinical parameters. In some embodiments, signal values include sensors data processed by processor 114. In some embodiments, output unit 122 is configured to output other indications such as alerts based on ranges defined for one or more clinical parameters. In some embodiments, output unit 122 is configured to output indications about the quality of sensors data and/or signal values.

The measuring device 100 may include a storage unit 112, configured to store one or more of the following data fields historical data received from the sensors, historical sensors activation instructions, and historical clinical data. In some embodiments, storage unit 112 stores a biological or medical condition of the patient, for example one or more or a combination of Acute HF, COPD level, infection (sepsis), pneumonia, sleep apnea, Hypertension, hypotension, fall, general deterioration. In some embodiments, storage unit 112 is used to store sensors data prior and/or after being processed by processor 114. In some embodiments, storage unit 112 is removable or allows to use a removable storage medium. In some embodiments, storage unit 112 is used to store the sensors activation rules. In some embodiments, storage unit 112 is used to store measurements plans used to define required measurements by the measuring device 100. In some embodiments, the sensors activation rules are adjusted to match a biological or medical condition of the patient. In some embodiments, storage unit 112 stores normal range of values of specific biological or medical parameters, and the electrical circuitry is configured to generate an alert in case one or more of the specific biological or medical parameters are outside the normal range of values.

In some embodiments, as shown for example in FIG. 1B, cardiac or cardiovascular sensors 104 have one or more sensing probes 106, such as a photoplethysmogram (PPG) sensor and an electrogram sensor. In some embodiments, photoplethysmogram (PPG) sensor has light sources and light detectors for detecting light reflections from a body tissue. The photoplethysmogram (PPG) sensor may control the transmitted light, for example by controlling the light spectrum and frequency. The PPG sensor may also analyze the returned signal by measuring the returned pulse shape and the tissue absorption. In some embodiments, as shown in FIG. 1B, light sources and/or light detectors are exposed to a body tissue via a tissue facing surface 101a of housing 102.

In some embodiments, measuring device 100 includes one or more thermometers for measuring one or more body regions. The body regions may be, for example core body, skin, near body regions and the like. The thermometer may include one or more sensors such as thermocouple, resistive temperature device, and infrared sensors.

In some embodiments, measuring device 100 includes one or more accelerometers measuring movement, vibration, and rotation in all axes.

In some embodiments, measuring device 100 includes one or more galvanic skin response (GSR) sensors between two or more contact points. In some embodiments, measuring device 100 includes one or more sensors for measuring electrical activity such as ECG, EMG and EEG.

Measuring device 100 may further comprise a power source 124 connected to the two or more sensors. In some embodiments, power source 124 has a measurable power capacity. In some embodiments, the sensors activation rules comprise activation intervals associated with the measured power capacity. For example, the activation frequency of at least one of the two or more sensors is reduced when the measured power capacity is reduced. In another example, the selection of a sensors to be activated is in accordance with the measured available power capacity. In some embodiments, power source 124 is a battery and measured power source is equivalent to the level of battery charging level.

According to some embodiments, switching circuit 116 is configured to generate activation signals to one or more sensors of the two or more sensors. In some embodiments, switching circuit 116 is activated by a source outside measuring device 100.

In some embodiments, output unit 122 is adapted to output clinical data of at least 1 minute of clinical parameters measurements. In some embodiments, output unit 122 is adapted to output clinical data of at least 5 minutes of clinical parameters measurements. In some embodiments, output unit 122 is adapted to output clinical data of at least 10 minute of clinical parameters measurements. Such clinical data output cycle may allow continuous measuring and monitoring of clinical parameters.

Measuring device 100, according to the invention, can be used for continues monitoring of one or more clinical parameters for a long period of time. The exemplary operational parameters provided elsewhere herein are for demonstrating the continuous monitoring. Some examples of the clinical parameter are systolic blood pressure, diastolic blood pressure, mean arterial pressure, pulse pressure, stroke volume, cardiac output, cardiac index, systemic vascular resistance, blood oxygen saturation, tissue oxygen saturation, respiratory rate, breathing volume, Heart rate, Heart rate variability (HRV), cardiac arrhythmia, level of sweat, movements, gait, calories consumption, body temperature, Hemoglobin level, glucose/sugar level, sleep quality, lactate, bilirubin level, fat level and a combination thereof.

According to some embodiments, measuring device 100 further includes a communication circuit adapted to communicate with one or more other devices (e.g., devices having similar functionality to the measuring device 100, or other connectable devices). In some embodiments, communication circuit 122 has a receiver circuit for receiving data from the one or more other measuring devices. In some embodiments, communication circuit 122 has a transmitter for transmitting data to the one or more other measuring devices, such data may be received and/or sent over a wireless medium, protocol or technique. According to some embodiments, electrical circuitry 110 has a calibration functionality adapted to receive data from the one or more other measuring devices, and to output calibration data. In some embodiments, the calibration functionality receives as an input calibration data of the one or more other measuring devices and processes the calibration data.

According to some embodiments, electrical circuitry 110 controls the operation of measuring device 100 (e.g., sensors switching circuit 116, processor 114, and output unit 120). In some embodiments, electrical circuitry 110 is configured to trigger measuring of clinical parameters during specific conditions evaluated based on signals received from the sensors. For example, during (i) Sepsis, when body temperature increases by set value and HR increased by another set value, (ii) Acute HF, when respiratory rate increased by set value and HR increased by another set value. In some embodiments, electrical circuitry 110 is configured to trigger measuring of clinical parameters during specific conditions evaluated based on signals received from one or more cardiac or cardiovascular sensors 104. In some embodiments, electrical circuitry 110 is configured to trigger measuring of clinical parameters during specific conditions evaluated based on signals received from one or more additional sensors 108. Some examples of such conditions are

Normal cardiac sinus rate is detected by an electrogram sensor (e.g., ECG), cardiac arrhythmia is detected by an electrogram sensor (e.g., ECG), Epileptic condition is detected by an electrogram sensor (e.g., EEG), Sleep stages like REM, non-REM, deep sleep, are detected by an electrogram sensor (e.g., EOG or EMG), and sweating periods as detected by an electrogram sensor that monitors changes in the electric activity of the tissue.

HR below or over set threshold, HRV below or above set threshold (e.g., lower than 40-60) BP values above or below set threshold (e.g., higher than 80-140). In some embodiments, measuring device 100 includes a setting functionality for setting one or more of above thresholds.

In another embodiment of the invention a clinical parameter measuring system is provided, including a first and a second measuring devices 100 as disclosed herein. In some embodiments, the first and second measuring devices 100 are configured to operate simultaneously. Some examples of simultaneous operation are exchanging of calibration data (e.g., during a set up period), activation of one measuring device when the other measuring device is not activated, improving clinical parameter value accuracy (e.g., by using median filter), measuring different ranges of clinical parameters values (e.g., during high SNR, low HR, or rest use), monitoring different clinical conditions (e.g., using ECG for HR detection otherwise use PPG), etc.

According to some embodiments, the one or more measuring devices 100 disclosed herein measure the following parameters simultaneously HR, HRV (Heart rate variability), BP, BPV (blood pressure variability), SV, CO, CI, SVR, RR, SAT%, skin and body temperature.

According to an aspect of the invention there is a method of measuring clinical parameters using one or more measuring devices disclosed herein.

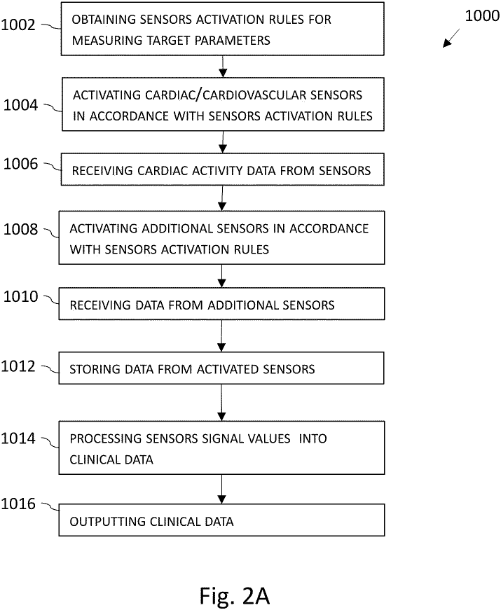

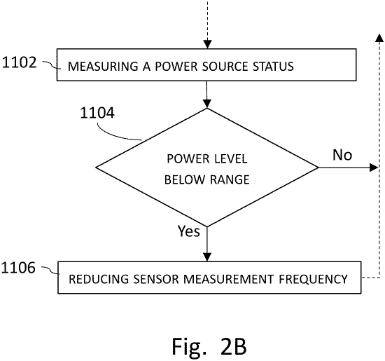

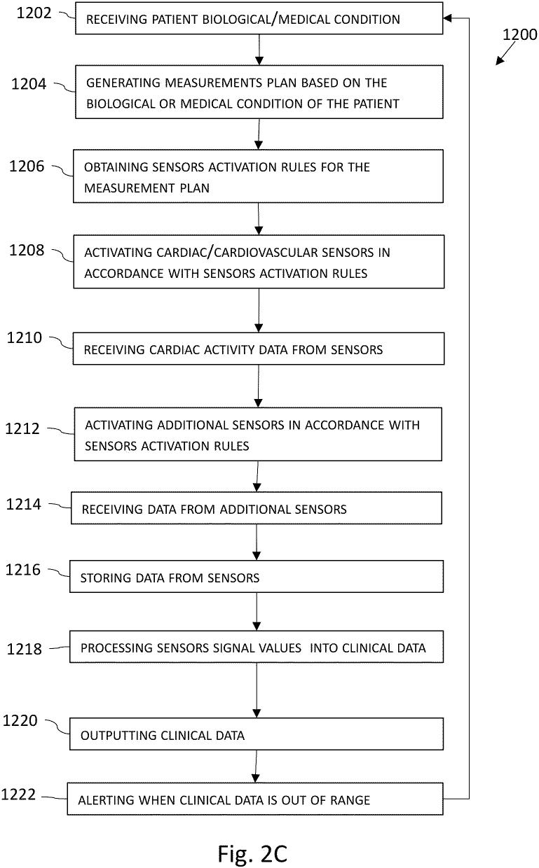

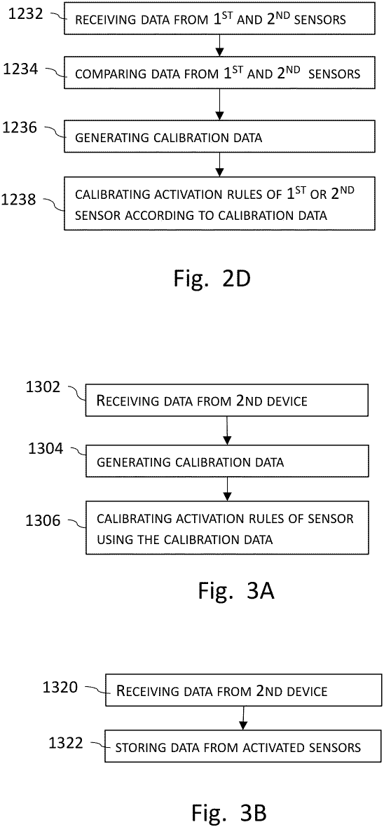

Reference is now made to FIGS. 2A to 2C which are flow diagrams of some examples of processes for measuring clinical parameters, according to some embodiment of the current invention. As shown in FIG. 2A, the process of measuring clinical parameters 1000 using a measuring device such as device 100 disclosed herein, the measuring process 1000 includes the following steps

Step 1002 discloses obtaining sensors activation rules for measuring parameters.

Step 1004 discloses activating cardiac and/or cardiovascular sensors, such as sensors 104 disclosed herein, in accordance with the sensors’ activation rules. According to some embodiments, activating the cardiac and/or cardiovascular sensors optionally includes measuring of the electrical activity of a tissue by one or more electrogram sensors included with the measuring device.

Step 1006 discloses receiving cardiac activity data from the sensors of the measuring device.

In some embodiments, receiving the cardiac activity data may be performed when systolic pressure is within or outside a target range, for example above 140 to 160 mmHg or below 80 to 100 mmHg. In some embodiments, receiving the cardiac activity data may be performed when diastolic pressure is within or outside a target range, for example above 90 to 110 mmHg, or below 60 to 80 mmHg. In some embodiments, receiving is performed when cardiac arrhythmia is detected by cardiac and/or cardiovascular sensors 104.

Step 1008 discloses activating additional sensors, such as sensors 108, in accordance with sensors activation rules. In some embodiments, activating steps 1004 and 1008 are performed by activating a total of two or more sensors in a continuous manner. In some embodiments, activating steps 1004 and 1008 are performed by activating a total of three or more sensors in a continuous manner. In some embodiments, activating steps 1004 and 1008 are performed by activating a total of five or more sensors in a continuous manner. In some embodiments, activating steps 1004 and 1008 includes an option to activate all sensors simultaneously, the cardiac and the additional sensors. In some embodiments, activating additional sensors 1008 is triggered based on data received from the cardiac and/or cardiovascular sensors.

Step 1010 discloses receiving data from the additional sensors of the measuring device.

Step 1012 discloses storing data from the sensors of the measuring device. In some embodiments, of the stored data is received from at least one of steps 1006 and 1010.

Step 1014 discloses processing sensors signal values into clinical data. Such processing may be performed by the measuring device. Such processing may be performed according to a set of rules stored in the memory of the measuring device.

Step 1016 discloses outputting the clinical data. In some embodiments, outputting 1016 is performed when the clinical data is within a pre-defined range.

According to some embodiments, one or more of steps 1002-1016 are performed repeatedly, for example once every 5 seconds during one week. Repeating may be for a continuous measurement of the clinical parameters. In some embodiments, steps 1002-1012 are repeated prior to the processing of step 1014. In some embodiments, steps 1002-1014 are repeated prior to the outputting clinical data of step 1016.

Steps 1002 to 1016 may be performed in a different order than disclosed above. For example processing 1014 and outputting 1016 may be performed prior to steps 1008 to 1010. Alternatively, steps 1008 to 1010 may be performed prior to steps 1004 and 1006.

FIG. 2B shows an optional method in which the measuring process 1000 may control sensors’ operation in accordance with available power source. In some embodiments, measuring process 1000 may further comprise the following steps

Step 1102 discloses measuring a power source status. In some embodiments, power source is a battery and measuring is of the battery charge level.

Step 1104 discloses determining if power level is below a predefined power range or threshold. If power level is below the predefined power range, step 1106 is performed, for reducing sensor measurement frequency of one or more of activated sensors. In some embodiments, reducing the sensor measurement frequency includes deactivating one or more sensors. If the power level is below the predefined power range, of the measuring device may generate an alert signal or message. The alert may be sent to an operator.

FIG. 2C shows steps of a process 1200 of measuring clinical parameters using a measuring device such as device 100 disclosed herein.

Step 1202 discloses receiving patient biological/medical condition. Some examples of the patient biological/medical condition include the following Acute HF, COPD level, infection (sepsis), pneumonia, sleep apnea, Hypertension, hypotension, fall, general deterioration. In some embodiments, receiving is by an input unit of the measuring device. In some embodiments, the patient biological/medical condition are received from a second remote device by a communication circuit of the measuring device.

Step 1204 discloses generating a measurements plan based on the biological or medical condition of the patient. In some embodiments, generating the measurements plan is performed using an input unit of the measuring device. In some embodiments, generating the measurements plan is performed by or on a remote device and received by the measuring device.

Step 1206 discloses obtaining specific sensors activation rules which are specific for the measurement plan. In some embodiments, the obtaining specific sensors activation rules are obtained from a storage unit within the measuring device. In some embodiments, the obtaining specific sensors activation rules are obtained from a remote device and received by the measuring device.

Step 1208 discloses activating cardiac and/or cardiovascular sensors, such as sensors 104 disclosed elsewhere herein, in accordance with the specific sensors activation rules which is specific to the measurement plan.

Step 1210 discloses receiving cardiac activity data from the sensors of the measuring device. In some embodiments, receiving the cardiac activity data may be performed when systolic pressure is within or outside a target range, for example above 140 to 160 mmHg or below 80 to 100 mmHg. In some embodiments, receiving the cardiac activity data may be performed when diastolic pressure is within or outside a target range, for example above 90 to 110 mmHg, or below 60 to 80 mmHg. In some embodiments, receiving is performed when cardiac arrhythmia is detected by cardiac and/or cardiovascular sensors 104.

Step 1212 discloses activating additional sensors, such as sensors 108, in accordance with sensors activation rules. In some embodiments, activating steps 1208 and 1212 are performed by activating a total of two or more sensors in a continuous manner. In some embodiments, activating steps 1208 and 1212 are performed by activating a total of three or more sensors in a continuous manner. In some embodiments, the measuring device may activate five or more sensors in a continuous manner. In some embodiments, activating steps 1208 and 1212 includes an option to activate all sensors simultaneously, the cardiac and the additional sensors. In some embodiments, activating the additional sensors is triggered based on data received from the cardiac and/or cardiovascular sensors.

Step 1214 discloses receiving data from the additional sensors of the measuring device.

Step 1216 discloses storing data from the sensors of the measuring device. In some embodiments, the stored data received from one or both of cardiac and/or cardiovascular sensors and the additional sensors.

Step 1218 discloses processing sensors signal values into clinical data.

Step 1220 discloses outputting the clinical data. In some embodiments, outputting 1220 is performed when the clinical data is within a pre-defined range.

Step 1222 discloses alerting when parameters values are out of a predefined normal range.

According to some embodiments, one or more of steps 1202-1222 are repeated. Repeating may result in a continuous measurement of the clinical parameters. In some embodiments, steps 1202-1216 are repeated prior to processing sensors signal values into clinical data. In some embodiments, steps 1202-1218 are repeated prior to outputting clinical data.

Steps 1202 to 1222 may be performed in a different order than disclosed above. For example processing 1218, outputting 1220 and or alerting 1222 may be performed prior to steps 1212 to 1214. Alternatively, steps 1212 to 1214 may be performed prior to steps 1208 to 1210.

Similar to process 1000, measuring process 1200 may include a functionality to control sensors’ operation in accordance with available power source.

According to some embodiments, the process of measuring 1000/1200 may comprise the steps of calculating a signal to noise ratio for one or more of the two or more sensors, and activating the one or more sensors based on the calculated signal to noise ratio.

According to some embodiments, the process of measuring 1000 may also comprise calibrating the measuring device. Calibrating may be performed before, in parallel, or after any one part of the measuring process 1000.

According to some embodiments, the calibration process follows a procedure of receiving calibration data by an electrical circuit provided in the measuring device. In some embodiments, receiving calibration data is performed from one or more sensors coupled to a body tissue. In some embodiments, the calibration data are received from one or more sensors of a wearable measuring device.

According to some embodiments, as shown for example in FIG. 2D, the clinical parameters measuring processes such as measuring 1000/1200 comprise calibrating using data of sensors within the measuring device. For example

Step 1232 discloses receiving data from a first sensor and a second sensor. Step 1234 discloses comparing data from the first sensor and the second sensor. Step 1236 of generating calibration data. Step 1238 of calibrating the sensors activation rules of the first and the second sensors using the generated calibration data.

In an alternative embodiment, as shown for example in FIG. 3A, calibrating of the measuring device is performed using data received from another device. The other device may be a second measuring device, such as device 100. The calibration process using another device may comprise the following steps

Step 1302 discloses receiving sensors data from a second device. Step 1304 discloses generating calibration data. Step 1306 discloses calibrating sensors activation rules using the generated calibration data.

In an alternative embodiment, as shown for example in FIG. 3B, measuring of clinical parameters such as measuring processes 1000 and/or 1200 is performed using two or more measuring devices, such as device 100, and includes

Step 1320 discloses receiving data from a second device and step 1322 discloses storing data from activated sensors in the second device.

According to some embodiments, measuring 1000/1200 is performed during specific medical conditions, such as Acute HF, COPD level, infection (sepsis), pneumonia, sleep apnea, Hypertension, hypotension, fall, general deterioration, cardiac arrhythmia, sepsis, shock or trauma, and bleeding. In some embodiments, measuring 1000/1200 is continuous during such medical conditions. According to some embodiments measuring 1000 is initiated when one or more of the following measured parameters are within a pre-defined range BP, HR, respiratory rate, HRV, pulse pressure, Mean arterial pressure, stroke volume, cardiac output, cardiac index, systemic vascular resistance, body temperature, blood oxygen saturation.

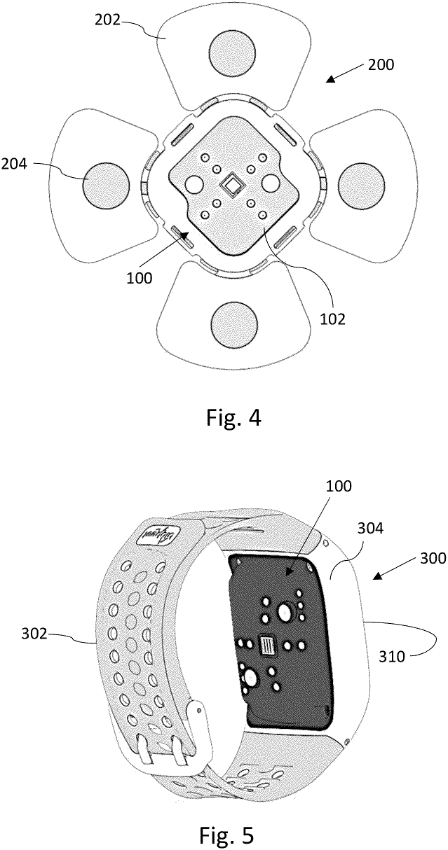

Reference is now made to FIG. 4 which illustrates a bottom view of a wearable apparatus for measuring clinical parameters, according to exemplary embodiments of the invention. As shown in FIG. 4, measuring device, such as device 100 described above, is wearable by having an attachment-case 200 for coupling device 100 to a tissue of a body organ (e.g., skin, limb or chest). In some embodiments, attachment-case 200 is undetachably attached to housing 102. In some embodiments, attachment-case 200 is attachable to the tissue by an adhesive layer 202. In some embodiments, attachment-case 200 is adapted to affix measuring device 100 to the tissue. In some embodiments, attachment-case 200 is adapted to affix measuring device 100 to the tissue by pressing tissue facing layer 101a of device 100 to the tissue.

As shown in FIG. 4. attachment-case 200 may further comprise one or more sensors or one or more electrodes 204. The one or more sensors or one or more electrodes 204 may be adapted to measure cardiac or cardiovascular activity. The one or more sensors or one or more electrodes 204 may be additional sensors which are not configured for measuring cardiac or cardiovascular activity. In some embodiments, the measurements of the one or more sensors or one or more electrodes 204 provide indication about the quality of connection between the measuring device 100 and the tissue of the body organ.

Attachment-case 200 may be shaped to extend diametrically away of a central point or central area of housing 102. In some embodiments, the one or more sensors or one or more electrodes 204 are located at a portion of attachment-case 200, which extend diametrically away of a central point or central area of housing 102. In some embodiments, adhesive layer 202 is extending away of housing 102.

In some embodiments, attachment-case 200 further includes a display unit (not shown). In some embodiments, attachment-case 200 further includes a display unit port (e.g., transparent window) allowing for indications provided by measuring device 100 (e.g., of a display unit of device 100) to be accessible by a user of the measuring device 100.

Referring now to FIG. 5 which illustrates an isometric view of a wearable measuring device, according to exemplary embodiments of the invention.

As shown in FIG. 5, wearable measuring device 300 has a securing module 302 in the form of a bracelet and a housing 304 coupled to securing module 302. In some embodiments, wearable device 300 is in the form of a watch-like device.

According to some embodiments, wearable measuring device 300 includes a measuring unit 100 which is structurally similar to the measuring device 100 embodiments disclosed herein. In some embodiments, measuring unit 100 is housed within housing 304. In some embodiments, securing module 302 further includes a display unit 310. In some embodiments, display unit 310 is coupled to housing 304.

According to some embodiments, measuring device 200 and/or 300 provides indications about the clinical parameters and/or the operational state of measuring device 200/300 using one or more of visual signals, voice signals, vibrations, and the like. In some embodiments, device 200 and/or 300 further includes a display unit which can show values related to the measured clinical parameters, e.g., values provided by output unit 120.

The terms “comprise”, “including”, “includes”, “including”, “has”, “having” and their conjugates mean “including but not limited to”. The term "consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure. As used herein, the singular forms “a”, “an” and “the” include plural references unless the context clearly dictates otherwise.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc., as well as individual numbers within that range, for example, 1, 2, 3, 4, 5, and 6. This applies regardless of the breadth of the range. Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween. Unless otherwise indicated, numbers used herein and any number ranges based thereon are approximations within the accuracy of reasonable measurement and rounding errors as understood by persons skilled in the art

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements. Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

What is claimed is:

1. A measuring device for measuring one or more clinical parameters of a patient, comprising:
a housing;
two or more sensors located in the housing, and comprising:
one or more cardiac or cardiovascular sensors for outputting cardiac activity values, having one or more sensing probes selected from a photoplethysmogram (PPG) sensor and an electrogram sensor;
one or more additional sensors, selected from a galvanic skin response (GSR) sensor, an accelerometer, and a thermometer, and
electrical circuitry located in the housing and electrically connected to the two or more sensors, the electrical circuitry comprising:
a storage unit for storing sensors data and sensors activation rules, wherein the sensors activation rules dictate which of the two or more sensors is used to sample the clinical parameters;
a processor configured to: process the sensors data; and
a sensors switching circuit configured to determine which sensors of the two or more sensors collect information in a given time frame in accordance with the sensors’ activation rules,
an output unit configured to receive signal values from the sensors and to output clinical data.

2. The device of claim 1, further comprising:
a measurements storage unit, configured to store historical data received from the sensors, historical sensors activation instructions and historical clinical data.

3. The device of claim 1, wherein the electrical circuitry is configured to activate the one or more cardiac or cardiovascular sensors and the one or more additional sensors when the device is set to measure the one or more clinical parameters.

4. The device of claim 1, further comprises an input unit for receiving or updating the sensors activation rules.

5. The device of claim 1, wherein the sensors activation rules further comprise sensors operational range, such as: measuring range, sensitivity, and signal filtering profile.

6. The device of claim 1, wherein the sensors activation rules further comprise target clinical parameters, such as: measuring range of values, clinical parameters to measure.

7. The device of claim 1, comprising:
a power source connected to the two or more sensors, and
the sensors activation rules comprise activation intervals associated with a measured power condition, such that activation intervals frequency of at least one of the two or more sensors is reduced when measured power source is reduced.

8. The device of claim 7, wherein the power source is a battery and the measured power source is equivalent to the level of battery charging level.

9. The device of claim 1, wherein the device is a wearable device having the housing couplable to a body organ.

10. The device according to claim 9, wherein the body organ is a limb or a chest.

11. The device of claim 1, further comprising a communication circuit adapted to communicate with one or more remote devices, and having a receiver circuit for receiving data from the one or more remote devices.

12. The device of claim 11, further comprising calibration functionality adapted to receive as an input data from the one or more remote devices, to processes calibration data and to output the calibration data.

13. The device of claim 1, wherein the electrical circuitry stores a biological or medical condition of the patient and rules for activating the sensors based on the biological or medical condition of the patient.

14. The device of claim 13, wherein the stored biological or medical condition comprise one or more of Acute HF, COPD level, infection (sepsis), pneumonia, sleep apnea, Hypertension, hypotension, fall, other cardio-pulmonary diseases, neurological diseases, psychological conditions, general deterioration and a combination thereof.

15. The device of claim 1 wherein the electrical circuitry selects a specific biological or medical parameter to be measured based on the biological or medical condition of the patient and activates the sensors in order to measure the selected specific biological or medical parameter.

16. The device of claim 15, wherein the specific biological or medical parameter comprise one or more of: systolic blood pressure, diastolic blood pressure, mean arterial pressure, pulse pressure, stroke volume, cardiac output, cardiac index, systemic vascular resistance, blood oxygen saturation, tissue oxygen saturation, respiratory rate, breathing volume, pulse rate, pulse rate variability, Heart rate, Heart rate variability (HRV), cardiac arrhythmia, level of sweat, movements, gait, calories consumption, body temperature, Hemoglobin level, glucose/sugar level, sleep quality, lactate, bilirubin level, fat level, and a combination thereof.

17. The device of claim 16, wherein the electrical circuitry stores allowed range or values of the specific biological or medical parameters; wherein the electrical circuitry generates an alert in case one or more of the specific biological or medical parameters are outside the allowed range or values.

18. The device of claim 1, wherein at least two of the two or more sensors collect information simultaneously regarding the same biological or medical parameter.

19. The device of claim 1, wherein determining which sensors of the two or more sensors will determine the value of the clinical parameter in a given time frame based on the patient’s condition and/or device operational status and/or the signals measured from the two or more sensors.

20. The device of claim 1, wherein one sensor of the two or more sensors is used to calibrate measurements of a second sensor of the two or more sensors.

21. The device of claim 1, wherein the sensor activation rules dictate that a first sensor of the two or more sensors is used to measure a specific biological or medical parameter when the measured values are in a first set range and a second sensor of the two or more sensors is used to measure the specific biological or medical parameter when the measured values are in a second set range.

22. The device of claim 1, comprising at least five sensors, wherein the at least five sensors comprising a photoplethysmogram (PPG) sensor, an electrogram sensor, a galvanic skin response (GSR) sensor, an accelerometer, and a thermometer.

23. The device of claim 22, wherein the at least five sensors collect information in a continuous manner.

24. The device of claim 22, wherein the at least five sensors collect information at set intervals.

25. The device of claim 24, wherein the set intervals depend on a status of a battery providing electrical power to the device.

26. The device of claim 1, wherein the clinical parameters comprise one or more of systolic blood pressure, diastolic blood pressure, mean arterial pressure, pulse pressure, stroke volume, cardiac output, cardiac index, systemic vascular resistance, blood oxygen saturation, tissue oxygen saturation, respiratory rate, breathing volume, Heart rate, Heart rate variability (HRV), cardiac arrhythmia, level of sweat, movements, gait, calories consumption, body temperature, Hemoglobin level, glucose/sugar level, sleep quality, lactate, bilirubin level, fat level, and a combination thereof.

27. The device of claim 1, wherein the electrical circuitry is configured to measure signal to noise ratio of at least one of the two or more sensors and selecting which of the sensors to be used based on the signal to noise ratio.

28. A method for measuring one or more clinical parameters of a patient by a measuring device having two or more sensors and sensors activation rules, the method comprising:
activating one or more cardiac and/or cardiovascular sensors which are part of the two or more sensors, according to the sensors” activation rules;
receiving cardiac activity data from the one or more cardiac and/or cardiovascular sensors;
activating one or more additional sensors which are part of the two or more sensors, according to the sensors’ activation rules;
storing data received from the activated sensors;
processing sensors signal values into clinical data by a computing unit located in the measuring device; and
outputting the clinical data.

29. The method of claim 28, comprising:
checking the power level in the power source; and
reducing the measurement frequency of one or more of the activated sensors if power level is below a predefined threshold.

30. The method of claim 28, further comprising:
receiving a biological or medical condition of the patient;
generating a measurement plan based on the biological or medical condition of the patient; and
obtaining sensors activation rules for the measurement plan.

31. The method of claim 28, further comprising:
calculating a signal to noise ratio for one or more of the two or more sensors; and
activating one or more sensors based on the calculated signal to noise ratio.

32. The method of claim 28, wherein the activating of one or more cardiac and/or cardiovascular sensors and one or more additional sensors is by activating three or more sensors in a continuous manner.

33. The method of claim 28, further comprising:
receiving data from a second measuring device; and
generating calibration data for the two or more sensors and/or the sensors activation rules.

34. The method of claim 28, further comprising:
receiving data from a first and a second sensors of the two or more sensors;
comparing between the values of measurements received from the first and the second sensors; and
generating calibration data for the two or more sensors and/or the sensors activation rules.

35. The method of claim 28, further comprising:
selecting clinical condition of the patient;
defining clinical parameters for monitoring by the measuring device; and
obtaining activation rules based on defined one or more clinical parameters for monitoring.

36. The method of claim 28, further comprising obtaining sensors activation rules based on the patient’s current and/or historical condition.

37. The method of claim 28, further comprising obtaining sensors activation rules based on device operational status and/or the signals measured from the two or more sensors.
